(19) United States
(12) Patent Application Publication
(10) Pub. No.: US 2026/0244812 A1
(43) Pub. Date: Aug. 20, 2026

(54) HIGH-CYCLE FATIGUE LIFE ANALYSIS METHOD BASED ON STATIC SIMULATION OF SPINNING PULLEY

(71) Applicant: Fuzhou University, Fuzhou City (CN)

(72) Inventors: Ligang YAO, Fuzhou City (CN); Juan Zheng, Fuzhou City (CN); Yingjie Cai, Fuzhou City (CN); Huiyi Yan, Fuzhou City (CN); Yangxin Chen, Fuzhou City (CN); Biaolin LUO, Fuzhou City (CN); Yaming LIU, Fuzhou City (CN)

(21) Appl. No.: 19/375,837

(22) Filed: Oct. 31, 2025

(30) Foreign Application Priority Data
Feb. 17, 2025 (CN) 202510172769.X

(51) Int. Cl. G06F 30/17

(52) U.S. Cl. CPC G06F 30/17

(57) ABSTRACT

Provided is a high-cycle fatigue life analysis method based on static simulation of a spinning pulley, including a modeling approach based on numerical simulation of actual working conditions of a spinning pulley using Ansys, where the modeling approach incorporates factors including rotational speed, fixed support, pressure, and standard Earth gravity of a spinning pulley model; in a constructed model, an axial load on the spinning pulley is converted into a pressure according to a formula, and the pressure is applied as a constraint during operation of the spinning pulley; an equivalent stress of the spinning pulley is obtained through Ansys simulation, an S-value in an S-N curve is derived via mean stress correction, an N-value is obtained from the S-N curve, and finally high-cycle fatigue life of the spinning pulley is calculated using a Miner's rule formula.

### CROSS REFERENCE TO RELATED APPLICATION

This patent application claims the benefit and priority of Chinese Patent Application No. 202510172769X, filed with the China National Intellectual Property Administration on Feb. 17, 2025, the disclosure of which is incorporated by reference herein in its entirety as part of the present application.

### TECHNICAL FIELD

The present disclosure relates to the technical field of model analysis, and in particular, to a high-cycle fatigue life analysis method based on static simulation of a spinning pulley.

### BACKGROUND

Pulleys are critical transmission components widely used in various mechanical equipment, including aerospace, engineering machinery, agricultural machinery, automobiles, and machine tools. To overcome the shortcomings of traditional manufacturing processes and achieve lightweight pulley designs, researchers worldwide have devoted significant efforts in recent decades to studying advanced pulley forming techniques—spinning technology. Pulleys produced using this technology offer advantages such as smooth transmission, high precision, material savings, high production efficiency, environmental friendliness, and long service life. Related technical research has long attracted widespread attention and yielded considerable scientific achievements. However, since spinning pulleys are subjected to alternating loads during power transmission and motion, the primary failure mode is fatigue fracture. Fatigue failure is highly concealed, often exhibiting no obvious deformation before rupture, which can lead to severe safety accidents and is difficult to prevent.

Currently, fatigue testing of spinning pulleys mainly involves suspending the pulley and rotating it until microcracks appear. Such fatigue tests are not only time-consuming, labor-intensive, and material-intensive but also involve lengthy measurement cycles. Therefore, some studies have taken spinning pulleys as the research subject, using limit stress diagrams to derive a safe fatigue operating range for plastic components under combined bending and shear deformation, as well as a calculation formula for a fatigue safety factor under complex stress states. These studies further compute the fatigue safety factor with the aid of finite element analysis results. The fatigue safety factor helps determine whether a pulley will experience fatigue failure, which is of great significance for assessing and measuring the fatigue life of the pulley.

### SUMMARY

The present disclosure provides a high-cycle fatigue life analysis method based on static simulation of a spinning pulley. The method includes: obtaining a stress-strain contour plot of a spinning pulley through Ansys static simulation, acquiring a stress-number of cycles (S-N) curve using nCode, performing mean stress correction via the Goodman equation, and finally calculating high-cycle fatigue life of the spinning pulley based on Miner's rule. By implementing this technical solution, the stress distribution of spinning pulleys under actual operating conditions can be visualized more intuitively, fatigue life of the spinning pulleys can be calculated with enhanced accuracy, and reliable reference data can be provided for developing spinning pulleys with superior fatigue service performance.

The present disclosure uses the following technical solutions.

A high-cycle fatigue life analysis method based on static simulation of a spinning pulley is provided, including a modeling approach based on numerical simulation of actual working conditions of a spinning pulley using Ansys, where the modeling method incorporates factors including rotational speed, fixed support, pressure, and standard Earth gravity of the spinning pulley; in a constructed model, an axial load on the spinning pulley is converted into a pressure according to a formula, and the pressure is applied as a constraint during operation of the spinning pulley; an equivalent stress of the spinning pulley is obtained through Ansys simulation, an S-value in an S-N curve is derived via mean stress correction, an N-value is obtained from the S-N curve, and finally high-cycle fatigue life of the spinning pulley is calculated using a Miner's rule formula.

The method further includes obtaining the S-N curve of a material using nCode and performing fatigue life calculations based on Miner's rule.

The method includes the following steps:
step S1: establishing a geometric model of the spinning pulley;
step S2: selecting the material and setting physical parameters of the material;
step S3: defining a gravitational acceleration, a pressure, fixed support, a rotational speed, and analysis settings of the spinning pulley under an actual working condition;
step S4: performing meshing and obtaining a Von Mises stress contour plot of the material;
step S5: obtaining the S-N curve of the material;
step S6: performing mean stress correction; and
step S7: calculating high-cycle fatigue life.

Step S1 includes the following steps:
step S1.1: creating a model of the spinning pulley using SolidWorks, where the spinning pulley has a maximum outer diameter of 192 mm, a minimum inner diameter of 87 mm, seven V-grooves, and two spring grooves, and a final physical model of the spinning pulley is as shown in FIG. 1;
step S1.2: after the model is created, importing the spinning pulley into Ansys Design Modeler to facilitate subsequent meshing and improve mesh quality, and drawing a circle having a diameter of 150 mm with a center point of the spinning pulley on an xy-plane as an origin;
step S1.3: using an extrusion command to divide the spinning pulley into two annular bodies; and
step S1.4: using a cutting command to divide the spinning pulley into four parts with a zx-plane as a reference, where the ultimate geometric model is as shown in FIG. 2.

Step S2 specifically includes: in Engineering Data of Ansys, customizing the material of the spinning pulley as low-carbon steel specifically steel grade 08AL; physical parameters required for the geometric model include density, Young's modulus, Poisson's ratio, yield strength, tensile strength, fatigue strength, and environmental temperature. Details are shown in Table 1.

| Material property | Unit | Value |
|---|---|---|
| Young's modulus | MPa | 207000 |
| Poisson's ratio | | 0.28 |
| Yield strength | MPa | 188 |
| Tensile strength | MPa | 270 |
| Fatigue strength | MPa | 135 |
| Environmental temperature | C. | 22 |

Step S3 specifically includes the following steps:
step S3.1: adding standard Earth gravity and setting the gravitational acceleration to 9806.6 mm/s²;
step S3.2: adding the pressure, where a pressure conversion formula is:

$$P = \frac{F}{S \times sin \frac{θ}{2}}$$

where F represents an axial load of the spinning pulley under the actual working condition (1800N), S represents a side area of the V-groove (65.8 cm²), and θ represents a V-groove angle (40°);
step S3.3: adding the fixed support, and selecting a large inner ring of the spinning pulley as a fixed support surface;
step S3.4: adding the rotational speed and setting the rotational speed to 1500 rad/s; and
step S3.5: setting the number of steps, current step number, initial substep, and minimum substep of a corresponding analysis process in Ansys to 1, setting step end time to 1 s, and setting maximum substep to 10.
Step S4 is specifically as follows:

step S4.1: selecting a sweeping method for an outer annular body of the spinning pulley, setting a sweep element size to a required value of 2 mm, and selecting quadrilateral/triangular as a free face mesh type;
step S4.2: selecting a patch conforming method for an inner annular body of the spinning pulley, adding geometric size adjustment, and adjusting an element size to a required value of 2 mm; and
step S4.3: after mesh generation, adding an equivalent stress command in a solution scheme command, and clicking the solution scheme command to obtain the Von Mises stress contour plot and a maximum equivalent stress value, as shown in FIG. 3.
Step S5 specifically includes:

customizing the S-N curve in nCode, where the S-N curve is defined as a two-segment curve, and input physical parameters include: material type, yield strength (YS), ultimate tensile strength (UTS), elastic modulus (E), stress range intercept (SRI1), slope of a first segment of the S-N curve (b1), transition point of the S-N curve (Nc1), slope of a second segment of the S-N curve (b2), standard deviation of the S-N curve (SE), stress ratio (RR), and cutoff limit of the S-N curve (Nfc), with details as shown in Table 2; and after inputting data of each physical parameter, obtaining the S-N curve of the material 08AL, as shown in FIG. 4.

| Material property | Unit | Value |
|---|---|---|
| MaterialType | | 99 |
| YS | Mpa | 188 |
| UTS | Mpa | 270 |
| E | Mpa | 2.07E5 |
| SRI1 | Mpa | 8945 |
| b1 | | −0.3333 |
| Nc1 | Cycles | 1E7 |
| b2 | | −0.2 |
| SE | N | 0 |
| RR | | −1 |
| Nfc | | 1E30 |

Step S6 specifically includes the following steps:
step S6.1: calculating a stress amplitude of the material under the actual working condition Sₐ and a mean stress Sₘ:

$${S}_{a} = \frac{( {S}_{m ax} - {S}_{m i n} )}{2}$$

$${S}_{m} = \frac{( {S}_{ma x} + {S}_{m i n} )}{2}$$

where Sₘₐₓ represents a maximum equivalent stress value in the Von Mises stress contour plot, and Sₘᵢₙ represents a minimum equivalent stress value in the Von Mises stress contour plot; and
step S6.2: performing mean stress correction using a Goodman equation to convert a stress amplitude at a stress ratio of Sₘᵢₙ/Sₘₐₓ to a stress amplitude Sn at a stress ratio of −1:

$$\frac{{S}_{a}}{{S}_{n}} + \frac{{S}_{m}}{{S}_{u}} = 1$$

where Sₐ represents the stress amplitude of the material under the actual working condition, Sₘ represents the mean stress, Sn represents the stress amplitude of the material under fully reserved cycle, and Su represents an ultimate tensile strength of the material.

Step S7 specifically includes the following steps:
step S7.1: setting the rotational speed and the axial load of the spinning pulley five times with different values to serve as five different working conditions, and obtaining corresponding S_{N1}, S_{N2}, S_{N3}, S_{N4}, and S_{N5} for each working condition through step S1 to step S6:
setting the rotational speed of the spinning pulley to 1,500 rad/s and the axial load to 1,800 N, and performing the aforementioned six steps to obtain S_{N1};
setting the rotational speed of the spinning pulley to 1,500 rad/s and the axial load to 2,500N, and performing the aforementioned six steps to obtain S_{N2},
setting the rotational speed of the spinning pulley to 1,500 rad/s and the axial load to 3,200N, and performing the aforementioned six steps to obtain S_{N3};
setting the rotational speed of the spinning pulley to 2,200 rad/s and the axial load to 1,800N, and performing the aforementioned six steps to obtain S_{N4};
setting the rotational speed of the spinning pulley to 3,000 rad/s and the axial load to 1,800N, and performing the aforementioned six steps to obtain SN₅;
step S7.2: based on the S-N curve in step S5, deriving N₁, N₂, N₃, N₄, and N₅ from S_{N1}, S_{N2}, S_{N3}, S_{N4}, and SN₅, respectively;
step S7.3: setting cycle counts of the spinning pulley under the five working conditions to n₁, n₂, n₃, n₄, and n₅, respectively; and
step S7.4: performing fatigue life calculations using Miner's rule:

$$\sum \frac{{n}_{i}}{{N}_{i}} = 1$$

where Nᵢ represents a cycle count for fatigue life of the spinning pulley under load Sᵢ, and nᵢ represents a cycle count of the spinning pulley under load Sᵢ

The present disclosure provides a high-cycle fatigue life analysis method based on static simulation of a spinning pulley, including a modeling approach based on numerical simulation of actual working conditions of a spinning pulley using Ansys. Considering factors including rotational speed, fixed support, pressure, and standard Earth gravity of the spinning pulley, a stress-strain contour plot of the spinning pulley is obtained through Ansys static simulation, an S-N curve is acquired using nCode, mean stress correction is performed via the Goodman equation, and ultimately high-cycle fatigue life of the spinning pulley is calculated based on Miner's rule. By implementing this technical solution, the stress distribution of spinning pulleys under actual operating conditions can be visualized more intuitively, fatigue life of the spinning pulleys can be calculated with enhanced accuracy, and reliable reference data can be provided for developing spinning pulleys with superior fatigue service performance.

The present disclosure is directed to improvements in the technical field of mechanical component design and analysis. Unlike purely mathematical calculations or theoretical models, the disclosed method integrates computer-aided static simulation of a physical spinning pulley with specific real-world operating conditions, such as rotational speed, fixed support, gravity, and pressure, to generate accurate fatigue life predictions. This provides a concrete technological improvement in the manufacture and use of pulleys, reducing the need for costly and time-intensive physical fatigue testing.

The disclosed fatigue life analysis method is not directed to an abstract mathematical formula in isolation. Instead, the method is explicitly applied to a physical pulley structure under defined loading conditions and material properties. The simulation environment incorporates actual constraints and forces experienced during pulley operation, ensuring that the output, namely fatigue life prediction, directly improves the design, durability, and safety of pulleys used in industrial applications.

By applying the Goodman equation and Miner's rule within a simulation-based environment tied to pulley geometries and real material characteristics, the disclosed method yields fatigue life data that can be directly integrated into manufacturing and engineering processes. This integration transforms the abstract calculations into a practical tool for engineering decision-making in the design and testing of pulleys.

Accordingly, the present disclosure is rooted in mechanical engineering technology and provides a specific improvement in pulley fatigue life prediction, which in turn enhances the safety and efficiency of pulley-driven mechanical systems.

### BRIEF DESCRIPTION OF THE DRAWINGS

The present disclosure will be further described in detail below with reference to the accompanying drawings and specific implementations:

FIG. 1 is a schematic diagram of a Solidworks model of a spinning pulley;

FIG. 2 is a schematic diagram of a DesignModeler model of a spinning pulley;

FIG. 3 is a schematic diagram of a Von Mises stress contour plot of a spinning pulley;

FIG. 4 is a schematic diagram of an S-N curve of a spinning pulley; and

FIG. 5 is a flow diagram of a high-cycle fatigue life analysis method based on static simulation of a spinning pulley.

### DETAILED DESCRIPTION OF THE EMBODIMENTS

As shown in the figures, a high-cycle fatigue life analysis method based on static simulation of a spinning pulley is provided, including a modeling approach based on numerical simulation of actual working conditions of a spinning pulley using Ansys, where the modeling method incorporates factors including rotational speed, fixed support, pressure, and standard Earth gravity of the spinning pulley; in a constructed model, an axial load on the spinning pulley is converted into a pressure according to a formula, and the pressure is applied as a constraint during operation of the spinning pulley; an equivalent stress of the spinning pulley is obtained through Ansys simulation, an S-value in an S-N curve is derived via mean stress correction, an N-value is obtained from the S-N curve, and finally high-cycle fatigue life of the spinning pulley is calculated using a Miner's rule formula.

The method further includes obtaining an S-N curve of the material using nCode and performing fatigue life calculations based on Miner's rule.

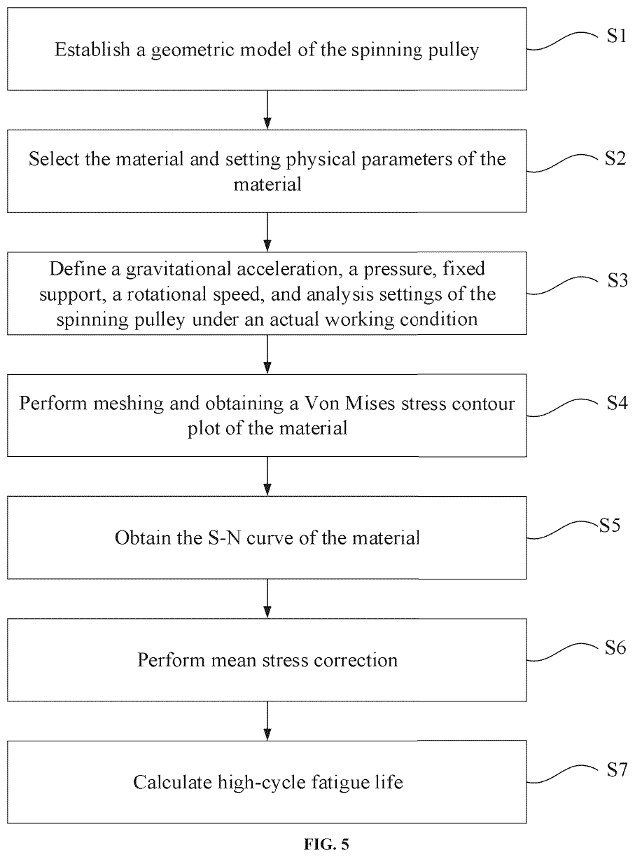

As shown in FIG. 5, the method includes the following steps:

Step S1: Establish a geometric model of the spinning pulley.

Step S2: Select the material and set physical parameters of the material.

Step S3: Define a gravitational acceleration, a pressure, fixed support, a rotational speed, and analysis settings of the spinning pulley under an actual working condition.

Step S4: Perform meshing and obtain a Von Mises stress contour plot of the material.

Step S5: Obtain the S-N curve of the material.

Step S6: Perform mean stress correction.

Step S7: Calculate high-cycle fatigue life.

Step S1 includes the following steps:

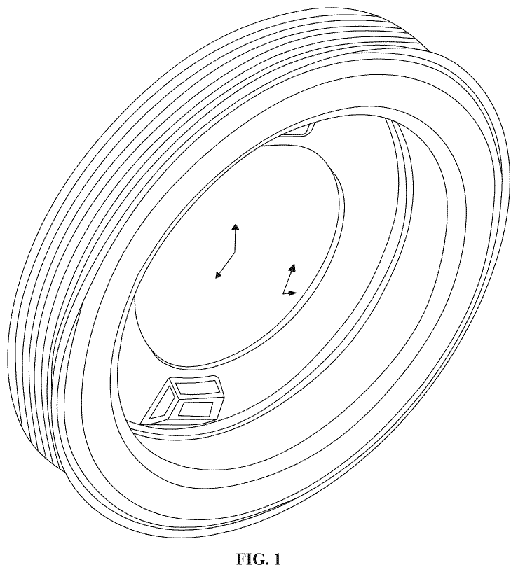

Step S1.1: Create a model of the spinning pulley using SolidWorks, where the spinning pulley has a maximum outer diameter of 192 mm, a minimum inner diameter of 87 mm, seven V-grooves, and two spring grooves, and a final physical model of the spinning pulley is as shown in FIG. 1.

Step S1.2: After the model is created, import the spinning pulley into Ansys Design Modeler to facilitate subsequent meshing and improve mesh quality, and draw a circle having a diameter of 150 mm with a center point of the spinning pulley on an xy-plane as an origin.

Step S1.3: Use an extrusion command to divide the spinning pulley into two annular bodies.

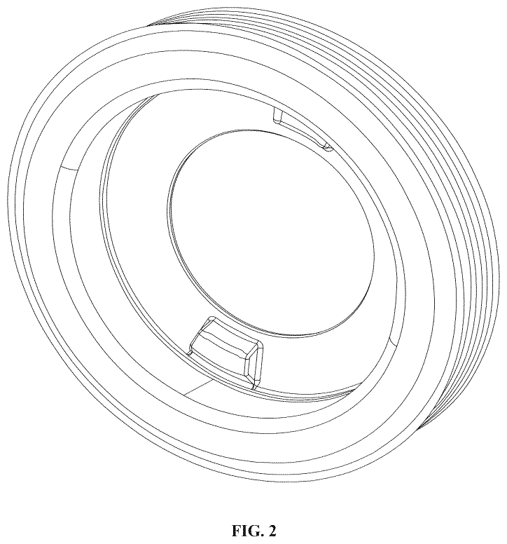

Step S1.4: Use a cutting command to divide the spinning pulley into four parts with a zx-plane as a reference, where the ultimate geometric model is as shown in FIG. 2.

Step S2 specifically includes: in Engineering Data of Ansys, customizing the material of the spinning pulley as low-carbon steel specifically steel grade 08AL; physical parameters required for the geometric model include density, Young's modulus, Poisson's ratio, yield strength, tensile strength, fatigue strength, and environmental temperature. Details are shown in Table 1.

| Material property | Unit | Value |
|---|---|---|
| Young's modulus | MPa | 207000 |
| Poisson's ratio | | 0.28 |
| Yield strength | MPa | 188 |
| Tensile strength | MPa | 270 |
| Fatigue strength | MPa | 135 |
| Environmental temperature | C. | 22 |

Step S3 specifically includes the following steps:

Step S3.1: Add standard Earth gravity and set the gravitational acceleration to 9806.6 mm/s².

Step S3.2: Add the pressure, where a pressure conversion formula is:

$$P = \frac{F}{S \times sin \frac{θ}{2}}$$

where F represents an axial load of the spinning pulley under the actual working condition (1800N), S represents a side area of the V-groove (65.8 cm²), and θ represents a V-groove angle (40°).

Step S3.3: Add the fixed support, and select a large inner ring of the spinning pulley as a fixed support surface.

Step S3.4: Add the rotational speed and set the rotational speed to 1500 rad/s.

Step S3.5: Set the number of steps, current step number, initial substep, and minimum substep of a corresponding analysis process in Ansys to 1, set step end time to 1 s, and set maximum substep to 10.

Step S4 is specifically as follows:

Step S4.1: Select a sweeping method for an outer annular body of the spinning pulley, set a sweep element size to a required value of 2 mm, and select quadrilateral/triangular as a free face mesh type.

Step S4.2: Select a patch conforming method for an inner annular body of the spinning pulley, add geometric size adjustment, and adjust an element size to a required value of 2 mm.

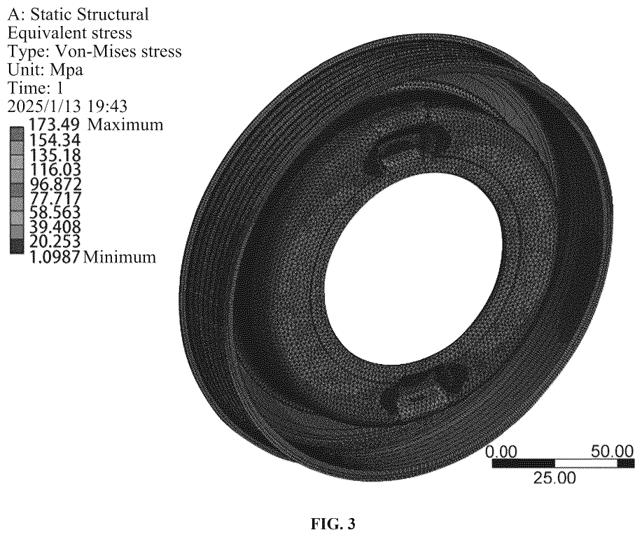

Step S4.3: After mesh generation, add an equivalent stress command in a solution scheme command, and click the solution scheme command to obtain the Von Mises stress contour plot and a maximum equivalent stress value, as shown in FIG. 3.

Step S5 specifically includes:

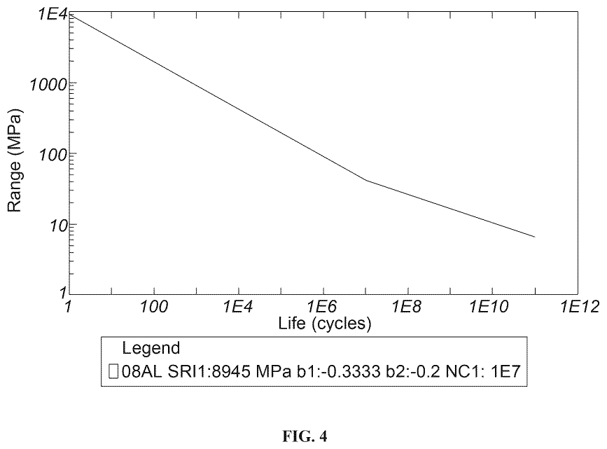

customizing the S-N curve in nCode, where the S-N curve is defined as a two-segment curve, and input physical parameters include: material type, yield strength (YS), ultimate tensile strength (UTS), elastic modulus (E), stress range intercept (SRI1), slope of a first segment of the S-N curve (b1), transition point of the S-N curve (Nc1), slope of a second segment of the S-N curve (b2), standard deviation of the S-N curve (SE), stress ratio (RR), and cutoff limit of the S-N curve (Nfc), with details as shown in Table 2; and after inputting data of each physical parameter, obtaining the S-N curve of the material 08AL, as shown in FIG. 4.

| Material property | Unit | Value |
|---|---|---|
| MaterialType | | 99 |
| YS | Mpa | 188 |
| UTS | Mpa | 270 |
| E | Mpa | 2.07E5 |
| SRI1 | Mpa | 8945 |
| b1 | | −0.3333 |
| Nc1 | Cycles | 1E7 |
| b2 | | −0.2 |
| SE | N | 0 |
| RR | | −1 |
| Nfc | | 1E30 |

Step S6 specifically includes the following steps:
Step S6.1: Calculate a stress amplitude of the material under the actual working condition Sₐ and a mean stress Sₘ:

$${S}_{a} = \frac{( {S}_{m ax} - {S}_{m i n} )}{2}$$

$${S}_{m} = \frac{( {S}_{ma x} + {S}_{m i n} )}{2}$$

where Sₘₐₓ represents a maximum equivalent stress value in the Von Mises stress contour plot, and Sₘᵢₙ represents a minimum equivalent stress value in the Von Mises stress contour plot.

Step S6.2: Perform mean stress correction using a Goodman equation to convert a stress amplitude at a stress ratio of Sₘᵢₙ/Sₘₐₓ to a stress amplitude Sn at a stress ratio of −1:

$$\frac{{S}_{a}}{{S}_{n}} + \frac{{S}_{m}}{{S}_{u}} = 1$$

where Sₐ represents the stress amplitude of the material under the actual working condition, Sₘ represents the mean stress, Sn represents the stress amplitude of the material under fully reserved cycle, and Su represents an ultimate tensile strength of the material.

Step S7 specifically includes the following steps:
Step S7.1: Set the rotational speed and the axial load of the spinning pulley five times with different values to serve as five different working conditions, and obtain corresponding S_{N1}, S_{N2}, S_{N3}, S_{N4}, and S_{N5} for each working condition through step S1 to step S6:

setting the rotational speed of the spinning pulley to 1500 rad/s and the axial load to 1800 N, and performing the aforementioned six steps to obtain S_{N1};
setting the rotational speed of the spinning pulley to 1500 rad/s and the axial load to 2500 N, and performing the aforementioned six steps to obtain S_{N2};
setting the rotational speed of the spinning pulley to 1500 rad/s and the axial load to 3200 N, and performing the aforementioned six steps to obtain S_{N3};
setting the rotational speed of the spinning pulley to 2200 rad/s and the axial load to 1800 N, and performing the aforementioned six steps to obtain S_{N4};
setting the rotational speed of the spinning pulley to 3000 rad/s and the axial load to 1800 N, and performing the aforementioned six steps to obtain S_{N5};
Step S7.2: Based on the S-N curve in step S5, deriving N₁, N₂, N₃, N₄, and N₅ from S_{N1}, S_{N2}, S_{N3}, S_{N4}, and S_{N5}, respectively.

Step S7.3: Set cycle counts of the spinning pulley under the five working conditions to n₁, n₂, n₃, n₄, and n₅, respectively.

Step S7.4: Perform fatigue life calculations using Miner's rule:

$$\sum \frac{{n}_{i}}{{N}_{i}} = 1$$

where Nᵢ represents a cycle count for fatigue life of the spinning pulley under load Sᵢ, and nᵢ represents a cycle count of the spinning pulley under load Sᵢ.

What is claimed is:

1. A high-cycle fatigue life analysis method based on static simulation of a spinning pulley, comprising a modeling approach based on numerical simulation of actual working conditions of a spinning pulley using Ansys, wherein the modeling approach incorporates factors comprising rotational speed, fixed support, pressure, and standard Earth gravity of the spinning pulley; in a constructed model, an axial load on the spinning pulley is converted into a pressure according to a formula, and the pressure is applied as a constraint during operation of the spinning pulley; an equivalent stress of the spinning pulley is obtained through Ansys simulation, an S-value in a stress-number of cycles (S-N) curve is derived via mean stress correction, an N-value is obtained from the S-N curve, and finally high-cycle fatigue life of the spinning pulley is calculated using a Miner's rule formula.

2. The high-cycle fatigue life analysis method based on static simulation of a spinning pulley according to claim 1, further comprising obtaining the S-N curve of a material using nCode and performing fatigue life calculations based on Miner's rule.

3. The high-cycle fatigue life analysis method based on static simulation of a spinning pulley according to claim 2, comprising the following steps:
step S1: establishing a geometric model of the spinning pulley;
step S2: selecting the material and setting physical parameters of the material;
step S3: defining a gravitational acceleration, a pressure, fixed support, a rotational speed, and analysis settings of the spinning pulley under an actual working condition;
step S4: performing meshing and obtaining a Von Mises stress contour plot of the material;
step S5: obtaining the S-N curve of the material;
step S6: performing mean stress correction; and
step S7: calculating high-cycle fatigue life.

4. The high-cycle fatigue life analysis method based on static simulation of a spinning pulley according to claim 3, wherein step S1 comprises the following steps:
step S1.1: creating a model of the spinning pulley using SolidWorks, wherein the model defines a maximum outer diameter, a minimum inner diameter, the number of V-grooves, and the number of spring grooves of the spinning pulley;
step S1.2: after the model is created, importing the spinning pulley into Ansys Design Modeler to facilitate subsequent meshing and improve mesh quality, and drawing a circle of a required diameter with a center point of the spinning pulley on an xy-plane as an origin;
step S1.3: using an extrusion command to divide the spinning pulley into two annular bodies; and
step S1.4: using a cutting command to divide the spinning pulley into four parts with a zx-plane as a reference, thereby obtaining the geometric model.

5. The high-cycle fatigue life analysis method based on static simulation of a spinning pulley according to claim 3, wherein step S2 specifically comprises: customizing the material of the spinning pulley as low-carbon steel in Engineering Data of Ansys, required physical parameters for the geometric model of the spinning pulley comprising density, Young's modulus, Poisson's ratio, yield strength, tensile strength, fatigue strength, and environmental temperature.

6. The high-cycle fatigue life analysis method based on static simulation of a spinning pulley according to claim 3, wherein step S3 specifically comprises the following steps:
step S3.1: adding standard Earth gravity and setting the gravitational acceleration;
step S3.2: adding the pressure, wherein a pressure conversion formula is:
$$P = \frac{F}{S \times sin \frac{θ}{2}}$$
wherein F represents an axial load of the spinning pulley under the actual working condition, S represents a side area of the V-groove, and θ represents a V-groove angle;
step S3.3: adding the fixed support, and selecting a large inner ring of the spinning pulley as a fixed support surface;
step S3.4: adding the rotational speed; and
step S3.5: setting the number of steps, current step, initial substep and minimum substep, step end time, and maximum substep for a corresponding analysis process in Ansys.

7. The high-cycle fatigue life analysis method based on static simulation of a spinning pulley according to claim 3, wherein step S4 specifically comprises:
step S4.1: selecting a sweeping method for an outer annular body of the spinning pulley, setting a sweep element size to a required value, and selecting quadrilateral/triangular as a free face mesh type;
step S4.2: selecting a patch conforming method for an inner annular body of the spinning pulley, adding geometric size adjustment, and adjusting an element size to a required value; and
step S4.3: after mesh generation, adding an equivalent stress command in a solution scheme command, and clicking the solution scheme command to obtain the Von Mises stress contour plot and a maximum equivalent stress value.

8. The high-cycle fatigue life analysis method based on static simulation of a spinning pulley according to claim 3, wherein step S5 specifically comprises:
customizing the S-N curve in nCode, wherein the S-N curve is defined as a two-segment curve, and input physical parameters comprise: material type, yield strength (YS), ultimate tensile strength (UTS), elastic modulus (E), stress range intercept (SRI1), slope of a first segment of the S-N curve (b1), transition point of the S-N curve (Nc1), slope of a second segment of the S-N curve (b2), standard deviation of the S-N curve (SE), stress ratio (RR), and cutoff limit of the S-N curve (Nfc); and after inputting data of each physical parameter, obtaining the S-N curve of the material.

9. The high-cycle fatigue life analysis method based on static simulation of a spinning pulley according to claim 3, wherein step S6 specifically comprises the following steps:
step S6.1: calculating a stress amplitude of the material under the actual working condition Sₐ and a mean stress Sₘ:
$${S}_{a} = \frac{( {S}_{m ax} - {S}_{m i n} )}{2}$$
$${S}_{m} = \frac{( {S}_{ma x} + {S}_{m i n} )}{2}$$
wherein Sₘₐₓ represents a maximum equivalent stress value in the Von Mises stress contour plot, and Sₘᵢₙ represents a minimum equivalent stress value in the Von Mises stress contour plot.
step S6.2: performing mean stress correction using a Goodman equation to convert a stress amplitude at a stress ratio of Sₘᵢₙ/Sₘₐₓ to a stress amplitude Sn at a stress ratio of −1:
$$\frac{{S}_{a}}{{S}_{n}} + \frac{{S}_{m}}{{S}_{u}} = 1$$
wherein Sₐ represents the stress amplitude of the material under the actual working condition, Sₘ represents the mean stress, Sn represents the stress amplitude of the material under fully reserved cycle, and Su represents an ultimate tensile strength of the material.

10. The high-cycle fatigue life analysis method based on static simulation of a spinning pulley according to claim 3, wherein step S7 specifically comprises the following steps:
step S7.1: setting the rotational speed and the axial load of the spinning pulley five times with different values to serve as five different working conditions, and obtaining corresponding S_{N1}, S_{N2}, S_{N3}, S_{N4}, and S_{N5} for each working condition through step S1 to step S6;
step S7.2: based on the S-N curve in step S5, deriving N₁, N₂, N₃, N₄, and N₅ from S_{N1}, S_{N2}, S_{N3}, S_{N4}, and S_{N5}, respectively;
step S7.3: setting cycle counts of the spinning pulley under the five working conditions to n₁, n₂, n₃, n₄, and n₅, respectively; and
step S7.4: performing fatigue life calculations using Miner's rule:
$$\sum \frac{{n}_{i}}{{N}_{i}} = 1$$
wherein Nᵢ represents a cycle count for fatigue life of the spinning pulley under load Sᵢ, and nᵢ represents a cycle count of the spinning pulley under load Sᵢ.
